(19) United States
(12) Patent Application Publication
(10) Pub. No.: US 2003/0086990 A1
(43) Pub. Date: May 8, 2003

(54) Method for treating carbon tetra-cloride induced liver damage by administering morinda citrifolia

(76) Inventors: Mian-Ying Wang, Rockford, IL (US); Claude Jarakae Jensen, Cedar Hills, UT (US); Chen Su, West Jordan, UT (US)

Correspondence Address: KIRTON AND MCCONKIE, 1800 EAGLE GATE TOWER, 60 EAST SOUTH TEMPLE, P O BOX 45120, SALT LAKE CITY, UT, 84145-0120, US

(21) Appl. No.: 10/036,152

(22) Filed: Dec. 31, 2001

Related U.S. Application Data
(60) Provisional application No. 60/335,314, filed on Nov. 2, 2001.

(51) Int. Cl. A61K 35/78

(52) U.S. Cl. 424/765; 424/769; 424/777

(57) ABSTRACT

There is disclosed a method for preventing CCL4 (carbon tetracloride) induced liver damage comprising the prophylactic administration of extracts of Morinda citrifolia. The Morinda citrifolia may be administered in solid or liquid forms. Several regimens are disclosed including the administration of 2 ounces twice daily in a liquid form such as that sold by Morinda, Inc.

### BACKGROUND

1. Field of the Invention

The present invention relates to methods and formulations for treating liver damage, and particularly to treating liver damage and preventing CCL4 (carbon tetracloride) induced liver damage comprising the prophylactic administration of extracts of Morinda citrifolia.

2. Background of the Invention

Liver damage from hepatitis C, alcohol, or carbon tetracloride is well documented. However, many of the treatments result in undesirable side effects.

Carbon tetrachloride (CC14) is a common environmental pollutant and liver carcinogen. CC14 is leached into the soil through agricultural run-off, spills, landfill contamination, and dumping illegal. Surface waters become contaminated due to industrial and agricultural activities, wastewater release, particularly from iron and steel manufacturing, as well as other major industries.

CC14 is used in alkalies, chlorine, industrial inorganic chemicals and in petroleum refining, agricultural chemicals, refrigerants, solvent for oils, fats, lacquers, varnishes, rubber waxes, rubber cement, resins, starting material in the manufacture of organic compounds cleaning agents for machinery, electrical equipment, and pharmaceuticals. In the past, it was used as a dry cleaning agent, a fire extinguisher, a grain fumigant, a pesticide, as well as a component of aerosol can propellants. CC14 gets in the air by industrial emission and evaporation from both soil and surface waters. The amount of CC14 has been increasing in the atmosphere in recent years because it is so stable in the troposphere. It has a residence time of 30 to 100 years. The most at-risk exposure groups are workers involved in the manufacture and use of CC14.

Over the last 50 years, carbon tetrachloride (CC 14) is the classic model compound used in the induction of liver injury and tumor. As a potent hepatotoxin, CC14 produces centrolobular necrosis, which causes liver damage. It has been widely accepted that the liver injury induced by CC14 depends upon its metabolism by cytochrome 2E 1 into the highly reactive form of the trichloromethyl (CC13) radicals that initiate lipid peroxidation of cell membrane. Others have suggested that active oxygen molecules, such as superoxide anion radicals (SAR), may plan an important role in the inflammation process after intoxication by CC14.

In order to protect the liver from environmental toxicants such as CC14, thousands of plants have been screened. Morinda citrifolia is an herb that has a wide range of medical properties, such as anti-cancer, anti-inflammatory, and detoxification. The major component in the Morinda citrifolia is proxeronine, named by Dr. Ralph Heinicki, which is a protein initiator and regulator. More than 100 other chemicals in different parts of the Morinda citrifolia tree have been identified. All of these compounds are beneficial to the human body. Data indicates that Morinda citrifolia is able to prevent cancer at the initiation stage of carcinogenesis by blocking carcinogen (DMBA)-induced DNA adduct formation, scavenging free radicals, quenching lipid hydroproxides, and selectively inhibiting COX-2.

The liver is an extremely vital organ that serves to metabolize carbohydrates and store them as glycogen, metabolize lipids (including cholesterol and certain vitamins), and proteins, manufacture bile, filter impurities and toxic material from the blood, produce blood-clotting factors, and destroy old, worn-out red blood cells. Certain reticuloendothelial cells (the Kupffer cells) play a role in immunity. These are able to regenerate themselves after being injured or diseased. If a disease progresses beyond the tissue's capacity to regenerate new cells, the body's entire metabolism is severely disturbed. Any number of disorders can affect the liver and interfere with the blood supply, the hepatic and Kupffer cells, and the bile ducts. The incredible complexity of liver chemistry and its fundamental role in human physiology is so daunting to researchers that the thought of simple plant remedies might have something to offer is both laughable and even insulting! This highlights again the limiting trap of the current research paradigm. Morinda citrifolia, a powerful antioxidant, anti-inflammatory nutritional supplement may possess a liver protection property.

### SUMMARY AND OBJECTS OF THE INVENTION

The present invention provides a prophylactic regimen to prevent damage to the liver when that damage results from disease or the side-effects of other treatments. The present invention features a method for preventing carbon tetracloride induced liver damage in mammals, inhibiting further liver damage in mammals, and preventing cancerous growth, in the liver of mammals, at the initiation stages of carcinogenesis by blockage of carcinogen-DNA adduct formation.

Morinda citrifolia is believed to have broad therapeutic effects including anticancer and anti-inflammatory activity. Experiments conducted by the inventors indicates that Morinda citrifolia possesses a cancer preventive effect at the initiation stage of carcinogenesis. In these studies, the protective effect of Morinda citrifolia on carbon tetrachloride (CC 14)-induced liver injury in female SD rats was examined. Twelve female SD rats were divided into two groups: placebo and Morinda citrifolia. Animals were supplied with 20% placebo or 20% Morinda citrifolia for 12 days, respectively. On the last day, three animals from each group were fed 0.25 ml/kg CC14. Another three animals were maintained as controls. All animals were sacrificed at 6 hours after CC14 treatment. Livers were removed for light microscopic (LM) and electron microscopic (EM) examination; superoxide anion radical (SAR) assay and lipid hydroperoxide (LPO) determination. Liver sections in placebo and Morinda citrifolia control groups demonstrated normal lobular architecture at the LM level and normal ultrastructure. Liver sections in the placebo+CC14 group showed acute liver damage at the LM: focal vacuolated, lipid-containing or necrotic hepatocytes surrounding central veins and focal inflammatory cells scattered throughout the lobule. There was a significant decrease in the number of swollen, lipid containing, and apoptotic hepatocytes in the Morinda citrifolia+CC14 group, compared to the placebo+CC14. At the EM level, glycogen depletion and lipid droplets in the cell plasma were observed in both CC14 treated groups. Swollen mitochondria, disorganization of RER with loss of ribosomes, and abundant focal areas of SER were scattered throughout the cytoplasm. Interestingly, Golgi complexes in placebo+CC14 group contain small low-density vesicles. Golgi complexes in the Morinda citrifolia+CC14 contain large vesicles with increased electron density, Golgi cisternal stacks were well developed, while those in the placebo+CC14 group were often swollen and diminished. Liver SAR and LPO levels in Morinda citrifolia+CC14 group were decreased to 50% and 20% of that in the placebo+CC14 group, respectively. In conclusion, Morinda citrifolia may protect liver from CC14 exposure by scavenging free radicals and blocking the lipid peroxidation.

### DETAILED DESCRIPTION OF THE PREFERRED EMBODIMENTS

It will be readily understood that the components of the present invention, as generally described and illustrated in the figures herein, could be arranged and designed in a wide variety of different configurations. Thus, the following more detailed description of the embodiments of the system and method of the present invention, and represented in FIGS. 1 through *, is not intended to limit the scope of the invention, as claimed, but is merely representative of the presently preferred embodiments of the invention.

It will be readily understood that the components of the present invention, as generally described herein, could be arranged and designed in a wide variety of different methods, configurations or formulations. Thus, the following more detailed description of the embodiments of the methods of the present invention, is not intended to limit the scope of the invention, as claimed, but is merely representative of the presently preferred embodiments of the invention.

The Indian Mulberry plant, known scientifically as Morinda citrifolia L., is a shrub, or small or medium sized tree 3 to 10 meters high. It grows in tropical coastal regions around the world. The plant grows in the wild, and it has been cultivated in plantations and small individual growing plots. The Indian mulberry plant has somewhat rounded branches and evergreen, opposite (or spuriously alternate), dark, glossy, wavy, prominently-veined leaves. The leaves are broadly elliptic to oblong, pointed at both ends, 10-30 cm in length and 5-15 cm wide.

The Indian mulberry flowers are small, white, 3 to 5 lobed, tubular, fragrant, and about 1.25 cm long. The flowers develop into compound fruits composed of many small drupes fused into an ovoid, ellipsoid or roundish, lumpy body, 5-10 cm long, 5-7 cm thick, with waxy, white or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged, 2-celled stones, each containing about 4 seeds.

When fully ripe, the fruit has a pronounced odor like rancid cheese. Although the fruit has been eaten by several nationalities as food, the most common use of the Indian mulberry plant was as a red and yellow dye source. Recently, there has been an interest in the nutritional and health benefits of the Indian mulberry plant.

Because the Morinda citrifolia fruit is for all practical purposes inedible, the fruit must be processed in order to make it palatable for human consumption and included in food products used to treat various ailments and diseases. Processed Morinda citrifolia juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other processes include freeze drying the fruit and juice. The fruit and juice can be reconstituted during production of the final juice product. Still other processes include air drying the fruit and juices, prior to being masticated.

In a currently preferred process of producing Morinda citrifolia juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and firmness. Spoiled and hard green fruit is separated from the acceptable fruit.

The ripened and aged fruit is preferably placed in plastic lined containers for further processing and transport. The containers of aged fruit can be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp. The juice can be filtered from the pulp.

The juice can be packaged into containers for storage and transport. Alternatively, the juice can be immediately processed into finished juice product. The containers can be stored in refrigerated, frozen, or room temperature conditions. The pulp can be blended in with the juice to make a puree. The Morinda citrifolia juice and puree can then be blended in a homogenous blend and mixed with other ingredients. The other ingredients consist of, but are not limited to water, fruit juice concentrates, flavorings, sweeteners, nutritional ingredients, botanicals, and colorings. The finished juice product is preferably heated and pasteurized at a minimum temperature of 181° F. (83° C.) or higher up to 212° F. (100° C.).

The product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

Pure juice can be processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, reverse osmosis filtration, or any other standard commercial filtration devices. The operating filter pressure preferably ranges from 0.1 psig up to about 1000 psig. The flow rate preferably ranges from 0.1 gpm up to 1000 gpm, and more preferably between 5 and 50 gpm.

In addition to the processing methods described above, other methods of processing fruit into oil product, fiber product, and juice product are contemplated and may be employed. Several embodiments of formulations of processed juice, oil, and fiber can be used.

It is believed that the many health benefits of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free xeronine, it contains appreciable amounts of the precursor of xeronine, called Proxeronine. Further, Morinda citrifolia contains the inactive form of the enzyme Proxeronase which releases Xeronine from proxeronine. A paper entitled, “The Pharmacologically Active Ingredient of Morinda citrifolia” by R. M. Heinicke of the University of Hawaii, indicates that Morinda citrifolia is “the best raw material to use for the isolation of xeronine,” because of the building blocks of proxeronine and proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell can not perform its job efficiently. Without pro-xeronine to produce xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from pro-xeronine, assists in enlarging the pores to allow better absorption of nutrients.

Each tissue has cells which contain proteins which have receptor sites for the absorption of xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

Because of its many benefits, Morinda citrifolia has been known to provide a number of anecdotal effects in individuals having cancer, arthritis, headaches, indigestion, malignancies, broken bones, high blood pressure, diabetes, pain, infection, asthma, toothache, blemishes, immune system failure, and others.

In one example, which is not meant to be limiting in any way, the beneficial Morinda citrifolia is processed into Tahitian Morinda citrifolia® juice manufactured by Morinda, Incorporated of Orem, Utah.

To practice the invention, Morinda citrifolia is administered to the patient exhibiting one or more of the signs of liver damage sufficient to eliminate or at least alleviate one or more of the signs or symptoms.

The preferred dosage is at least two ounces of Morinda citrifolia liquid administered twice daily. Greater doses do not create side effects and have been found beneficial. For example, in one embodiment, up to one liter was administered daily with a significant prophylactic effect and no side effects. Some anecdotal evidence also seems to indicate that remedial benefits may also be experienced.

Through several clinical experiments, it has been found that Morinda citrifolia is capable of treating liver damage, inhibiting the proliferation of further cell deterioration within the liver, and even preventing cancer at the initiation stages of carcinogenesis by blockage of carcinogen-DNA adduct formation. The following examples illustrate the results obtained from these experiments and are for illustrative purposes only. These are not meant to be limiting in any way as one ordinarily skilled in the art will recognize the various parameters and control groups that may be used to carry out the intended function of the present invention as intended herein.

### EXAMPLE ONE

Morinda citrifolia possesses a cancer preventive effect at the initiation sage of carcinogenesis by preventing carcinogen-DNA adduct formation, scavenging oxygen free radicals, quenching lipid peroxides, selectively inhibiting COX II and anti-inflammatory activity. Consuming 10% Morinda citrifolia in drinking water for seven days was able to reduce DMBA-induced DNA adducts by 70% in the liver of male C57 BL/6 mice. Our recent studies indicated that Morinda citrifolia is the strongest antioxidant among the four tested well-known antioxidants including Vitamin C, pycnogenol, and grape seed powder. The antioxidant activity of TNJ shows a dose-dependent effect against superodixe anion radicals (SAR) and lipid hydroperoxides (LPO) in vitro. These results indicate that Morinda citrifolia is a strong antioxidant. The latest data shows that Morinda citrifolia is a selective inhibitor of COX II in vitro. We already have reported positive data demonstrating liver protection using Morinda citrifolia from our preliminary study. Pre-consuming 10% and 20% TNJ in drinking water for 12 days was able to protect liver from CC14 exposure in female SD rats.

We hypothesize that Morinda citrifolia may possess a liver protective effect in a liver injury model induced by CC14 in vivo.

The aim of one experiment was to determine the preventive effect of TNJ on the acute liver injury induced by CC 14 in female SD rats and to examine the therapeutic effect of Morinda citrifolia on the damaged liver induced by CC14 in female SD rats.

The experiment was designed to determine the preventive effect of Morinda citrifolia against CC 14 induced hepatotoxicity and the therapeutic effect of Morinda citrifolia on the damaged liver induced by CC14 in female SD rats. Five-week old, 80-100 g virgin female SD rats were purchased from Harlan Sprague Dawley, Inc. (Indianapolis, Ind.), and housed in the animal facility.

Dose-dependent liver damage-induced by CC14: Animals were exposed to CC14 at 0.25, 0.5, and 1.0 ml/kg. The degree of liver damage caused by different doses of CC14 were examined by light and electron microscopy. Time-dependent liver injury induced by CC14: Animals were sacrificed at 0, 1, 3, 6, 9, 12, 16, and 24 hours after 0.5 mg/kg CC14 administration. The time dependent CC14-induced liver injury was examined by light microscopy.

Preventive effect of TNJ on the liver injury induced by CC14 and Prevention or delay of the onset of CC 14-induced liver injury: Preventive effect of TNJ on liver injury induced by CC14 was examined at different time points after 0.5 mg/kg CC14 administration by pretreating animals with 10% Morinda citrifolia compared with that of 10% placebo. Forty-eight female SD rats were divided into two groups: Placebo and Morinda citrifolia group. Animals in these two groups received 10% placebo, or 10% Morinda citrifolia, respectively. After 12 days, 24 rats from each group intragastrically received CC14 0.25 ml/kg. Three rats were sacrificed from each group at 0, 1, 3, 6, 9, 12, 16, and 24 hours after CC14 administration. The liver was removed for the examination by LM, EM, and for other biomarkers.

Dose-dependent protection of Morinda citrifolia: Twenty-seven rats were divided into seven groups, three animals of each group received water, 5%, 10%, 20%, 50% Morinda citrifolia or placebo for 12 days, respectively. After 12 days, all the animals received CC15 0.25 ml/kg for 6 hours. Then all the animals were sacrificed and the liver was removed for examination of LM, EM, and other biomarkers.

Based upon the results of different doses of CC14 treatment and different length of CC14 exposure times, we were able to choose an optimum exposure time as our positive liver damaged model, such as six or nine hours time points after CC14 administration. We treated those sick animals with damaged livers by using different doses of Morinda citrifolia, such as 5%, 10%, 20%, and 50% in drinking water to observe the healing processing of CC14 at different time points. The same doses of placebo were supplied to the sick animals as positive controls. The healing process was monitored by LM and EM examination. Liver functions were monitored before TNJ treatment and at the end of experiment. The results were compared with the placebo-treated animals.

Biomarkers Selected in this Project:

1. Superoxide anion radical (SAR) level in target organ-liver will be tested by TNB asay.

2. Lipid hydroperoxide (LPQ) level in target organ-liver will be tested by LMB assay.

3. CC14-induced liver damage and the protection by Morinda citrifolia will be observed by light microscopic observation on the cellular and tissue levels.

4. Electron microscopic observation of CC14-induced liver damage and the protection of TNJ a the ultrastructure level.

5. Immunochemistray staining for expression of signal transduction signals, such as COX II, COX I, and other markers.

All examined parameters were compared between the different groups. The preventive effect of Morinda citrifolia and the therapeutic effect on the CC14-induced liver injury model will be evaluated based upon the changes of the examined biomarkers between Morinda citrifolia and placebo groups. The correlation between LPO and SAR levels in liver were evaluated. It was found that the levels were higher in the CC 14 group than in the control placebo group. Accordingly, other biomarkers were compared. The histological examination showed that a protective effect of Morinda citrifolia on the centrollubelous necrosis induced by CC14. The antioxidant effect and liver protective effects were evaluated carefully. A comparison was made between Morinda citrifolia and placebo groups. The student T test was selected to estimate the significance between the different groups of this experiment.

In this experiment, findings indicated that juice made from Morinda citrifolia fruits might prevent cancer at the initiation stage of carcinogenesis by blockage of carcinogen-DNA adduct formation, a strong antioxidant activity, and selective COX-2 inhibition. Consumption of 10% Morinda citrifolia juice for 7 days was able to prevent 70% of DMBA-DNA adducts in the liver of mice. In this study, the protective effect of Morinda citrifolia juice on the liver injury induced by carbon tetrachloride (CC14) in female SD rats was examined by light and electron microscopy. Animals were supplied with 10% placebo or 10% Morinda citrifolia juice for 12 days, respectively. On the last day, each animal was fed 0.5 ml/kg of CC14. Animals were sacrificed at 0, 1, 3, 6, 9, 12, 16, and 24 hours after CC14 administration. Another three animals were supplied with water as normal controls. Livers were removed for light and electron microscopic (LM and EM) examinations. Liver sections in placebo, water, and Morinda citrifolia juice control groups demonstrated normal lobular architecture at the LM level and normal ultrastructure. Liver sections in the placebo+CC14 group showed acute liver damage at the LM level in a time-dependent manner. Changes included a gradual increase of focal cellular vacuolization, lipid-containing or necrotic hepatocytes surrounding central veins, and focal inflammatory cells that were scattered throughout the lobules at 24 hours. Acute liver damage in the Morinda citrifolia juice+CC14 group was significantly delayed and there was a decrease in the number of swollen, lipid containing, and apoptotic hepatocytes compared to that of the placebo+CC14 group at different time points. At the EM level, glycogen depletion and lipid droplets in the cell plasma were observed in both CC14 treated groups. Swollen mitochondria, disorganization of RER with loss of ribosomes, and abundant focal areas of SER scattered throughout the cytoplasm were demonstrated in the placebo+CC14 group. Interestingly, golgi complexes in the Morinda citrifolia juice+CC14 group contained large electron dense vesicles with well-developed cistemal stacks, while golgi in the placebo+CC14 group contained small low-density vesicles and the cistemal stacks were often swollen and diminished. These observations coincided with the decrease of liver superoxide anion radicals and lipid hydroperoxide levels in the Morinda citrifolia+CC14 group when compared to the placebo+CC14 group (decreased 50% and 80%, respectively).

### EXAMPLE TWO

In an additional experiment, a high dose of Morinda citrifolia juice (20%) showed significant decrease in liver injury when induced by a lower dose of CC 14 (0.25 ml/kg). In conclusion, this is the first finding of hepatic protection by Morinda citrifolia juice on acute liver injury induced by CC 14, indicating that Morinda citrifolia may protect the liver from carcinogen exposure. Therefore, it may prevent cancer at the initiation stage of hepatic carcinogenesis.

The present invention may be embodied in other specific forms without departing from its spirit of essential characteristics. The described embodiments are to be considered in all respects only al illustrative and not restrictive. The scope of the invention is, therefore, indicated by the appended claims, rather than by the foregoing description. All changes which come within the meaning and range of equivalency of the claims are to be embraced within their scope

What is claimed and desired to be secured by Letters Patent is:

1. A method for preventing carbon tetracloride induced liver damage in mammals, said method comprising the steps of:
administering, to a patient, a formulation comprising processed Morinda citrifolia.

2. The method of claim 1, wherein said Morinda citrifolia is administered in dosages comprising at least two ounces of processed Morinda citrifolia juice twice daily on an empty stomach for a period of at least two months.

3. The method of claim 1, wherein said Morinda citrifolia is in juice form.

4. The method of claim 1, wherein said Morinda citrifolia is in solid form.

5. The method of claim 1, wherein said Morinda citrifolia is in powder form.

6. The method of claim 1, wherein

7. The method of claim 1, wherein said formulation comprises said Morinda citrifolia in an amount between about 0.1 and 5 percent by weight, said formulation being administered for a period of at least seven days.

8. The method of claim 1, wherein said formulation comprises said Morinda citrifolia in an amount between about 5 and 10 percent by weight, said formulation being administered for a period of at least seven days.

9. The method of claim 1, wherein said formulation comprises said Morinda citrifolia in an amount between about 10 and 15 percent by weight, said formulation being administered for a period of at least seven days.

10. The method of claim 1, wherein said formulation comprises said Morinda citrifolia in an amount between about 15 and 20 percent by weight, said formulation being administered for a period of at least seven days.

11. The method of claim 1, wherein said formulation comprises said Morinda citrifolia in an amount between about 20 and 25 percent by weight, said formulation being administered for a period of at least seven days.

12. The method of claim 1, wherein said formulation comprises said Morinda citrifolia in an amount between about 25 and 30 percent by weight, said formulation being administered for a period of at least seven days.

13. The method of claim 1, wherein said formulation comprises said Morinda citrifolia in an amount between about 30 and 35 percent by weight, said formulation being administered for a period of at least seven days.

14. The method of claim 1, wherein said formulation comprises said Morinda citrifolia in an amount between about 35 and 40 percent by weight, said formulation being administered for a period of at least seven days.

15. The method of claim 1, wherein said formulation comprises said Morinda citrifolia in an amount between about 40 and 45 percent by weight, said formulation being administered for a period of at least seven days.

16. The method of claim 1, wherein said formulation comprises said Morinda citrifolia in an amount between about 45 and 50 percent by weight, said formulation being administered for a period of at least seven days.

17. The method of claim 1, wherein said formulation comprises said Morinda citrifolia in an amount between about 50 and 55 percent by weight, said formulation being administered for a period of at least seven days.

18. The method of claim 1, wherein said formulation comprises said Morinda citrifolia in an amount between about 55 and 60 percent by weight, said formulation being administered for a period of at least seven days.

19. The method of claim 1, wherein said formulation comprises said Morinda citrifolia in an amount between about 60 and 65 percent by weight, said formulation being administered for a period of at least seven days.

20. The method of claim 1, wherein said formulation comprises said Morinda citrifolia in an amount between about 65 and 70 percent by weight, said formulation being administered for a period of at least seven days.

21. The method of claim 1, wherein said formulation comprises said Morinda citrifolia in an amount between about 70 and 75 percent by weight, said formulation being administered for a period of at least seven days.

22. The method of claim 1, wherein said formulation comprises said Morinda citrifolia in an amount between about 75 and 80 percent by weight, said formulation being administered for a period of at least seven days.

23. A method of inhibiting further liver damage in mammals, said method comprising the steps of:
administering, to a patient, a formulation comprising processed Morinda citrifolia.

24. The method of claim 23, wherein said formulation comprises ten percent said Morinda citrifolia, said formulation being administered for a period of at least seven days.

25. The method of claim 23, wherein said formulation comprises said Morinda citrifolia in an amount between about 0.1 and 80 percent by weight, said formulation being administered for a period of at least seven days.

26. A method for preventing cancerous growth, in the liver of mammals, at the initiation stages of carcinogenesis by blockage of carcinogen-DNA adduct formation, said method comprising the steps of:
administering, to a patient, a formulation comprising processed Morinda citrifolia.

27. The method of claim 26, wherein said formulation comprises said Morinda citrifolia in an amount between about 0.1 and 80 percent by weight, said formulation being administered for a period of at least seven days.
